(19) United States
(12) Patent Application Publication
(10) Pub. No.: US 2021/0092821 A1
(43) Pub. Date: Mar. 25, 2021

(54) Remote Control For Lights Using Power Wiring

(71) Applicant: Huot Instruments. LLC, Menomonee Falls, WI (US)

(72) Inventors: Richard D. Ward, Menomonee Falls, WI (US); Peter D. Joseph, Mukwonago, WI (US)

(21) Appl. No.: 17/032,723

(22) Filed: Sep. 25, 2020

Related U.S. Application Data
(60) Provisional application No. 62/905,743, filed on Sep. 25, 2019.

(51) Int. Cl. H05B 47/185; A61B 90/30

(52) U.S. Cl. CPC H05B 47/185; A61B 90/35; A61B 90/30

(57) ABSTRACT

A remote operational control system for a medical lighting device is provided that employs the wiring carrying the power to the lighting elements of the device for carrying the remote operational control signals for the lighting device in the form of alterations in the baseline voltage transmitted along the wiring. The remote operational control system additionally does not radiate any radio frequency energy, making the system compatible within hospital environments, and employs high level data security.

### CROSS-REFERENCE TO RELATED APPLICATION

This application claims priority from U.S. Provisional Patent Application Ser. No. 62/905,743, filed on Sep. 25, 2019, the entirety of which is expressly incorporated herein by reference for all purposes.

### FIELD OF THE DISCLOSURE

The present disclosure relates generally to lighting controls, and more specifically to accessories for remotely controlling lighting.

### BACKGROUND OF THE DISCLOSURE

There are many ways to produce a remote control for a medical lighting system, such as a lighting device supported by and articulating support arm, for example, the Galaxy Ceiling Mounted Light sold by StarTrol® of Menomonee Falls, Wis. In one prior art configuration, these lighting systems are operated by a control box disposed on a wall within the room in which the lighting device is located. The control box includes a number of switches or buttons corresponding to the various operational characteristics of the lighting device. Through the use of the control box an individual can power on or off and vary the operation of the lighting device in order to have the lighting device deliver the desired amount and intensity of light from the lighting element.

While this configuration does provide the ability to control the operation of the lighting device remotely from the device, this configuration has significant disadvantages, particularly with regard to articulating lighting devices that are utilized in the medical industry to illuminate medical examination rooms and operating theaters, for example. These types of lighting devices include a number of rotation and/or articulating joints in the support arm the extends between a support attached to a wall or ceiling in the room, and the lighting element disposed opposite the support. In order to enable power and control wiring to be disposed within the support arm and enable the operation and control of the lighting elements on the lighting device. However, it is highly desirable to keep the size of the articulating support arm as small as possible in order to maximize the ability of the support arm to be moved (larger components of the support arm can negatively affect the range of motion available). Therefore, the addition of the wiring for the control box into the support arm in addition to the wiring present to power the lighting elements, the additional wires to be routed from the control box to the lighting elements necessitates the use of more slip rings in the sections of the support arm in order to transmit the control signals to the lighting elements.

To address these issues, an alternative prior art configuration system is a wireless control system that utilizes radio frequencies broadcast between a remote transmitter and a receiver on the lighting device that is operably connected to the lighting elements. The use of the receiver connected to the lighting elements allows the lighting device control system to be positioned and operated apart from the support arm, such that no additional components are required to be added to an existing lighting device other than the receiver being connected to the lighting element(s).

However, the radio frequency control signals transmitted by the remote transmitter to the receiver in the wireless system can be easily distorted, which degrades the reliability of the operation of the wireless control system. Specifically, in a hospital setting these types of wireless systems can be difficult to certify as an additional power supply is required for the transmitter, which can potentially interfere with other medical monitoring equipment in the room where the lighting device and the remote control system is being utilized.

Therefore, it is desirable to develop a remote control system for operating medical lighting devices that overcomes these issues with the prior art.

### SUMMARY OF THE DISCLOSURE

According to one aspect of an exemplary embodiment of the invention, a remote control system for a medical lighting device is provided that employs the wiring carrying the power to the lighting elements of the device for carrying the remote control signal for the lighting device. In this manner, the remote control system of the present disclosure can be employed with both existing and new manufactured lighting devices, as no additional components are required to be place within the support arm for the medical lighting device.

According to another aspect of an exemplary embodiment of the invention, the use of the existing power wiring of the medical lighting device by the remote control system additionally does not radiate any radio frequency energy when the remote control system is operated. Thus, the remote control system does not generate interference with its operation and is compatible within hospital environments, and employs high level data security including but not limited to, parity checking control information and repetitive transmission of control data.

These and other aspects, features and advantages of the invention will be made apparent from the following detailed description taken together with the drawing figures.

### BRIEF DESCRIPTION OF THE DRAWINGS

The drawings illustrate the best mode currently contemplated of practicing the present invention.

In the drawings:

FIG. 1 is a perspective view of a medical lighting device including a remote operational control system according to an exemplary embodiment the invention; and

FIG. 2 is a schematic view of the remote operational control system of FIG. 1.

### DETAILED DESCRIPTION OF THE DRAWINGS

Before the present compositions, apparatuses and methods are described, it is understood that this invention is not limited to the particular embodiments and methodology, as these may vary. It is also to be understood that the terminology used herein is for the purpose of describing particular exemplary embodiments only, and is not intended to limit the scope of the present invention which will be limited only by the appended claims.

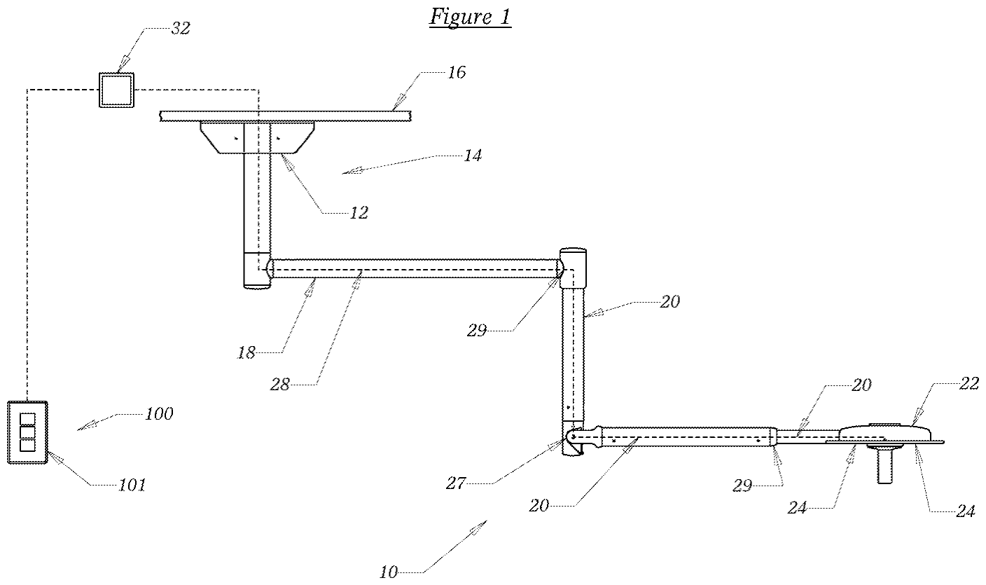

With reference now to FIG. 1 an exemplary embodiment of a medical lighting device is illustrated generally at 10. The device 10 includes a mount 12 secured to a support surface 14, such as a ceiling 16, though the surface 14 can additionally be a wall within the examination room or operating theater in which the device 10 is located, among other suitable environments. The device 10 additionally includes a support arm 18 extending outwardly from the mount 12. The support arm 18 can have multiple articulating sections 20 and supports a lighting housing 22 opposite the mount 12. The articulating sections 20 enable the lighting housing 22 to be positioned where desired within a wide range of locations relative to the support surface 14. For example, in addition to the mount 12 optionally being rotatable with regard to the support surface 14, each articulating section 20 is constructed such that adjacent sections 20 can rotate and optionally pivot with regard to one another. The sections 20 can additionally be configured to extend in horizontal and vertical directions with regard to one another, such that the lighting housing 22 can be vertically spaced from the support surface 14 in addition to the vertical spacing provided by the mount 12. In other additional embodiments, the sections 20 can be connected via hinged connections 27 with regard to one another, such that the sections 20 can be angularly displaced with regard to each other. Further, in other alternative embodiments, certain sections 20 can be constructed to be telescopic and/or slidable with regard to adjacent sections 20. The telescopic sections 20 enable the device 10 to be constructed with fewer sections 20 in the support arm 18 while providing adjustment for the device 10 over the same horizontal and/or vertical distance. The sliding sections 20 can provide the same horizontal and/or vertical adjustability while also reducing the number of sections 20 required to form the support arm 18. The hinged, telescoping and/or sliding sections 20 can also be provided with suitable locking mechanisms (not shown) that operate to secure the sections 20 in the desired configuration or position with regard to one another.

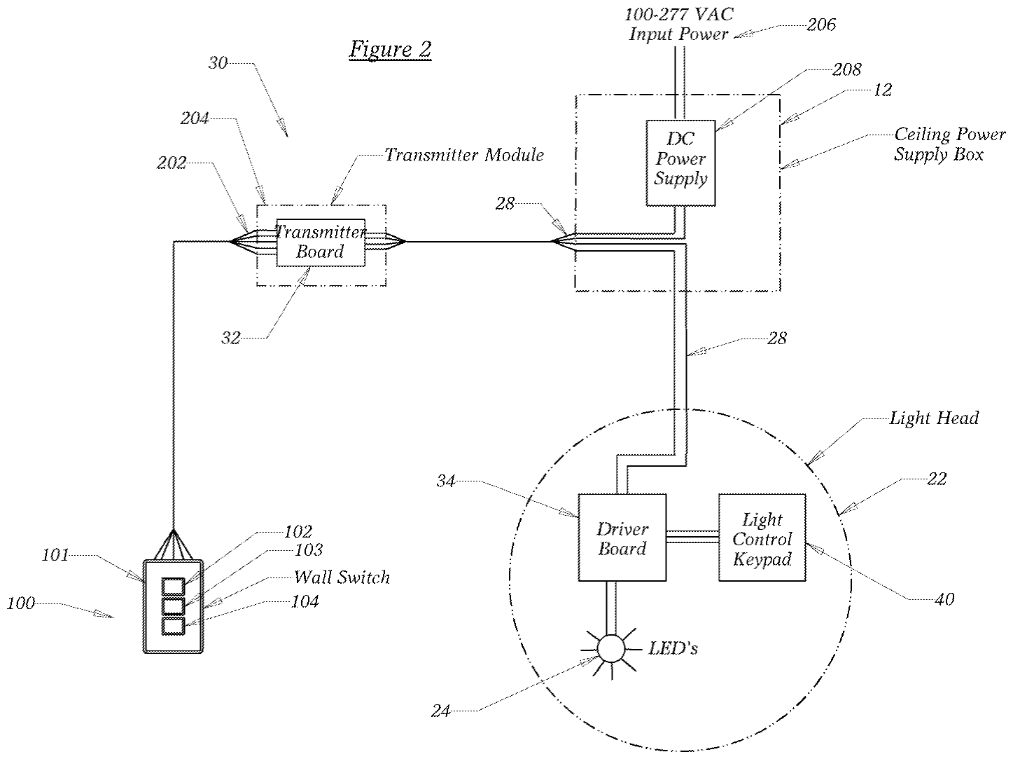

Referring now to FIGS. 1 and 2, opposite the support arm 18, the lighting housing 22 includes a number of light emitting elements or devices 24 to provide illumination once the sections 20 of the support arm 18 are moved to locate the lighting housing 22 where desired. In order to provide power to the light element 24, the device 10 includes wiring 28 disposed within the support arm 18 that extends between the individual light elements 24 and a suitable power supply 200 for the building or area in which the support surface 14 is located. The wiring 28 within the support arm 18 is configured in a manner that enables the wiring 28 to supply power to the light elements 24 regardless of the position of the sections 20 forming the support arm 18. For example, the sections 20 of the support arm 18 can each be formed with a number of slip rings 29 disposed at the connections between the adjacent sections 20. The slip rings 29 allow for electrical connections between rotating member or sections 20 to provide a path for the continuous supply of electrical power to the light element 24 from the power source via the wiring 28 connecting the slip rings 29 disposed on each section 20.

The controls for the operation of the light elements are contained within a remote operational control system 30 that is disposed in an accessible position within the room or theater in which the device 20 is placed. The remote operational control system 30 is operably connected to the medical lighting device 10 in order to selectively control the operation of the light elements 24 on the lighting housing 22, collectively, in specified groups or individually. The remote operational control system 30 has a control housing 100 including a number of control elements 101 in the form of switches, buttons, or the like that can be manually or optionally remotely operated to control the operational condition of the light element(s) 24 on the device 10. In the illustrated exemplary embodiment, a driver printed circuit board (PCB) 34 is disposed within the lighting housing 22 and operably connected to the lighting element(s) 24 and to the control housing 100 by the power wiring 28, in order to control the supply of power to the light elements 24 on the lighting housing 22. The control housing 100 includes a power switch 102 that, when activated, causes the driver PCB 34 to enable power from the power supply to flow along the wiring 28 to the light elements 24 in an on/off manner.

In order to control the light elements 24 in a manner beyond simply switching the light elements 24 on and off, the remote lighting control system 30 additionally includes a transmitter printed circuit board (PCB) 32 connected to corresponding receiver circuitry (not shown), operably connected to/disposed on or within the driver PCB 34, via the power wiring 28. The transmitter PCB 32 is disposed on the power wiring between the power supply 200 and the driver PCB 34 and sends control signals and other information from the PCB 32 over the existing power wiring 28 connecting the driver PCB 34 to the lighting element 22 in response to the operation of other buttons or switches 103,104 disposed on the control housing 100. The signals from the transmitter PCB 32 serve to selectively modify the power output from the power supply 200 to the driver PBC 34 to the lighting elements 24 to consequently adjust the operation of the light elements 24.

In an exemplary embodiment, the control housing 100/switch PCB includes a first on/off power switch 102, a second switch 103 for modifying the operation of the lighting elements 24 in a first manner, and a third switch 104 for modifying the operation of the lighting elements 24 in a second manner. The control housing 100/switch PCB is operably connected to the transmitter PCB 32, which can be disposed on or within in the mount 12, as in the exemplary illustrated embodiment, with suitable wired connections, that in the exemplary illustrated embodiment include a plug 202 engaged with a receptacle 204 on the transmitter PCB 32.

In operation, initially the switch or button 102 is activated to enable the switch PCB to signal the driver PCB 34, shown disposed within the light housing 22, to initiate the connection of the power supply 200 to the light elements 24 along the wiring 28. Upon further activation of switch 103 or 104 on the control housing 100, the signal from the switch PCB to the transmitter PCB 32 corresponding to the activation of the switch 103 or 104 causes transmitter PCB 32 to send a control signal over the power line 28 to the driver PCB 34 in a serial bit stream format. In order to enable the transmitter PCB 32 to accurately send the signal, the remote operational control system 30 is isolated in a known manner against varying power supply voltages and load conditions that would otherwise interfere with the clarity of the modified signal produced and sent over the power wiring 28 by the transmitter PCB 32.

To transmit the appropriate control signal over the power line 28 connecting the transmitter PCB 32 and the driver PCB 34, the transmitter PCB 32 receives and modifies the voltage along the power wiring 28 in manner that represents the control signal in manner recognizable to the driver PCB 34. In one exemplary embodiment of the present disclosure, for the control signal the transmitter PCB 32 encodes a “1” bit onto the voltage sent from the power supply 200, which in the illustrated exemplary embodiment includes a power converter 208 connected to an AC power supply 206, coming into the transmitter PCB 32 on the power supply line/wiring 28. More specifically, the transmitter PCB 32 accomplishes this by altering/dropping the supply voltage on the supply line/wiring 28 by 0.7 volts for 1 bit time. The drop in voltage representing the bit is negative, e.g., the 0.7V drop, as it is desirable to avoid the need to detect negative voltages, and as the reference voltage is positive (5.0V on the power supply line 28), a negative drop still results in a positive power voltage on the supply line 28. However, it is also possible reference the bit for the control signal by a positive voltage applied to the supply line wiring 28. In another example, the space condition or “0” bit can be set at 3.0 volts, i.e., an unaltered power supply line value, while a mark condition for a “1” bit can 2.3 volts, though this value can be modified as necessary or desired as long as the value is readily distinguishable from the unaltered voltage for the power supply. The absolute value of the voltage from the power supply can vary, but in an exemplary embodiment the voltage alteration for data signals of 0.7 volts and the voltage signal sense threshold of 0.3 volts, both negative to the supply rail/supply voltage, enable the signals to be encoded on the power supply wiring 28 in a manner that does not negatively affect the supply of power to the lighting elements 24 while allowing for clear signal indications to be transmitted with/along the power supply wiring 28. As a result, the operation of the remote operational control system 30 is not sensitive to power supply variations and accuracy. In an exemplary embodiment, the standard bit time for the “1” bit is set to 200 μs or 5000 baud, though other times can be utilized, to facilitate fast transmission times and stable voltage levels along the wiring 28.

The power supply modification signals/bits are assembled into a data stream composed of individual eleven (11) bit packets. Each packet includes a start bit, a data byte, a parity bit and a stop bit. In an exemplary embodiment, the data bytes are set either to an ASCII “U” for an up command, a “D” for a down command, or a “P” for a power command. Other commands and/or formats for the commands can be used or added as needed depending upon the functionality desired for the remote control system 30 The commands, or data bytes within the packets, are only sent when a button 103,104 is pressed on the remote control housing 100 that is operably connected to the transmitter PCB 32.

The receiver circuit/driver PCB 34 is AC coupled and referenced (shifted) to the internal +5V power supply line 28 between the PCB 34 and each of the light element(s) 24 that provides the voltage to the light element(s) 34 for their operation. As a result of the referenced signal, the received signal levels are now 5.0 volts for a “0” signal and 4.3 volts for a “1” signal in the bits within the signal packet. Consequently, in this particular exemplary embodiment, the receive signal threshold is set to about 4.7V In other exemplary embodiments, the “1” bit signal can unaltered, i.e., 0.7V, or can be set at about 0.3 volts less than the +5V rail 28, or about ½ the voltage of the 1″ bit signal from the transmitter PCB 32, which maintains a good threshold/differential between a “0” bit signal and a “1” bit signal.

On the circuit board for/within the driver PCB 34 an inverting comparator (not shown) or other suitable signal modifier is utilized to eliminate noise from the incoming signal, such as by using about 3% hysteresis in a known manner in an exemplary embodiment. The received signals are now set at 0.0 volts for a “0” bit and 5.0 volts for a “1” bit. These signals in the bit stream are passed within the driver PCB 34 to a conventional universal asynchronous receiver/transmitter (UART) (not shown) located on/within the driver PCB 34 to decode the bit signals contained within the transmitted serial packets and perform the operations corresponding to the bit signals. Packets with a bad parity calculation or commands (corrupted or omitted data bytes) that are not recognized are rejected.

These bit signals and data packets forming the commands are initiated by the operation of the switches 103,104 at the housing or panel 100, and generated by the above modification of the power supply line voltage and passed to the receiver/microcontroller/driver PCB 34 to set various lighting levels for the lighting element(s) 24 individually and/or collectively on the lighting device 10, as shown by the individual connections between the driver PCB 34 and the light elements 24 in FIG. 2. Further, the light housing 22 includes a keypad 40 thereon that is connected to the driver PCB 34 and enables the user to directly alter the operation of the PCB 34, as desired.

By slight alterations in the voltage carried along the power supply wiring 28 between the power source and the lighting elements 24 created by the transmitter PCB 32, and decoded by the microcontroller/receiver/driver PCB 34 attached to the lighting device 10, the operation of the lighting elements 24 can be controlled using only these small voltage variations in the power supply voltage transmitted over the existing power wiring 28 for the lighting device 10, enables a low cost, reliable remote lighting control system 20 that sends and detects of data/control signals for enhanced control of the lighting device 10 without interfering with the normal power transmission between the power source and the lighting element(s). In addition, by enabling the control signal to be transmitted by the transmitting PCB 32 over the existing power transmission line/wiring 28, the remote lighting control system 20 enables the placement and connection of the transmitter PCB 32 in the ceiling mount 22 with connection to the driver PCB 34 in the lighting housing 22 in newly manufactured or existing lighting devices 10.

Various other embodiments of the present invention are contemplated as being within the scope of the filed claims particularly pointing out and distinctly claiming the subject matter regarded as the invention.

We claim:

1. A remote operational control system for a medical lighting device, the system comprising:
a. a medical lighting device including at least one light element connected to a power source transmitting a supply voltage to the at least one light element along power supply wiring extending between the power source and the at least one light element;
b. a driver printed circuit board (PCB) operably connected to the power supply wiring to selectively enable the supply voltage to pass from the power source to the at least one light element; and
c. a transmitter PCB operably connected to the power supply wiring between the power source and the driver PCB, the transmitter PCB configured to send light element control data to the driver PCB along the power supply wiring.

2. The remote operational control system of claim 1 wherein the light element control data is in the form of alterations in the supply voltage along the power supply wiring.

3. The remote operational control system of claim 2 wherein the alterations in the supply voltage comprise drops in the supply voltage.

4. The remote operational control system of claim 3 wherein the alterations in the supply voltage comprise 0.7V drops in the supply voltage.

5. The remote operational control system of claim 2 wherein the alterations in the supply voltage each comprise a control signal bit.

6. The remote operational control system of claim 5 wherein each control signal bit has a signal duration of approximately 5000 baud.

7. The remote operational control system of claim 5 wherein an number of control signal bits are combined to form a bit packet.

8. The remote operational control system of claim 7 wherein the bit packet comprises a start bit, a data byte, a parity bit and a stop bit.

9. The remote operational control system of claim 8 wherein the data byte is selected from an up command, a down command, and a power command.

10. The remote operational control system of claim 1 wherein the light element control data is exclusively transmitted over the power supply wiring.
